(19) United States
(12) Patent Application Publication
(10) Pub. No.: US 2023/0235025 A1
(43) Pub. Date: Jul. 27, 2023

(54) METHOD FOR THE TREATMENT OF VIRUS INFECTION WITH IVIG AND CONVALESCENT PLASMA

(71) Applicant: GRIFOLS WORLDWIDE OPERATIONS LIMITED, DUBLIN (IE)

(72) Inventors: Elsa MONDOU, Dublin (IE); Rhonda GRIFFIN, Dublin (IE); Mireia TORRES HURTADO, Dublin (IE); Jordi NAVARRO PUERTO, Dublin (IE)

(21) Appl. No.: 17/999,370

(22) PCT Filed: May 26, 2021
(86) PCT No.: PCT/EP2021/064002

§ 371 Date: Nov. 18, 2022

Related U.S. Application Data
(60) Provisional application No. 63/030,698, filed on May 27, 2020.
(60) Provisional application No. 63/030,652, filed on May 27, 2020.

(51) Int. Cl. C07K 16/06; A61K 41/10; A61K 35/16; A61P 31/14

(52) U.S. Cl. CPC C07K 16/06; A61K 41/10; A61K 35/16; A61P 31/14; C07K 2317/76; C07K 2317/10; C07K 2317/21

(57) ABSTRACT

Method for the treatment of virus infection with IVIG and convalescent plasma. Methods and compositions for the treatment of coronavirus disease 2019 (COVID-19) in a patient in need thereof, including administering to the patient a therapeutically effective amount of intravenous Immunoglobulin G (IVIG) in an amount of about 0.5 g/kg to about 8 g/kg. Methods and compositions for the treatment of coronavirus disease 2019 (COVID-19) in a patient in need thereof, including administering to the patient a therapeutically effective amount of convalescent anti-SARS-CoV-2 plasma, wherein the convalescent anti-SARS-CoV-2 plasma is treated with methylene blue for pathogen inactivation.

The present disclosure is related to the field of pharmaceutical products. In particular, the present application refers to methods and compositions for the treatment of coronavirus disease 2019 (COVID-19) in a patient in need thereof, the method comprising administering to the patient a therapeutically effective amount of intravenous Immunoglobulin G (IVIG). The present application also refers to methods and compositions for the treatment of COVID-19 in a patient in need thereof, the method comprising administering to the patient a therapeutically effective amount of methylene blue treated (MBT) plasma from donors recovered from coronavirus disease 2019 (COVID-19).

Immunoglobulin G (IgG) is the isotype of the most abundant immunoglobulin in human serum (8-16 mg/ml), comprising approximately 80% of all immunoglobulins. IgG is indicated for the treatment of various diseases such as primary immunodeficiency, in particular congenital agammaglobulinaemia and hypogammaglobulinaemia, idiopathic thrombocytopenic purpura, as an adjuvant in the treatment of Kawasaki's Disease and in the transplant of bone marrow, hypogammaglobulinaemia associated with chronic lymphocyte leukaemia as part of the treatment of HIV infection in paediatric patients, among others.

At the present time there is high demand for IgG which is polyvalent with a wide spectrum of human antibodies and has total functionality (neutralising capacity, opsonisation, average life conserved), with intact molecules (integrity of the crystallisable Fc fragment) and a normal distribution of IgG subclasses identical or equivalent to natural plasma, especially for the minority subclasses (IgG3 and IgG4).

The routes for the therapeutic administration of IgG may be intravenous, subcutaneous and intramuscular, and in addition to this it may be administered by other less conventional routes such as the oral, inhaled or topical routes.

Nevertheless intravenous administration offers the most useful therapeutic indications, whether for the treatment of primary immunodeficiencies or for variable common immunodeficiency (deficit of IgG and IgA subclasses) (Espanol, T. “Primary immunodeficiencies”. Pharmaceutical Policy and Law 2009; 11(4): 277-283), secondary or acquired immunodeficiencies (for example infection by viruses such as cytomegalovirus, herpes zoster, human immunodeficiency) and diseases of an autoimmune origin (thrombocytopenic purpura, Kawasaki's Syndrome, for example) (Koski, C. “Immunoglobulin use in management of inflammatory neuropathy”. Pharmaceutical Policy and Law 2009; 11(4): 307-315).

Coronaviruses are a large family of positive-sense single-stranded RNA viruses which may cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS). The most recently discovered coronavirus, SARS-CoV-2, causes the associated coronavirus disease COVID-19. This new virus and disease were unknown before the outbreak began in Wuhan, China, in December 2019.

The most common symptoms of COVID-19 are fever, tiredness, and dry cough. Some patients may have aches and pains, nasal congestion, runny nose, sore throat, or diarrhea. These symptoms are usually mild and begin gradually. Some people become infected but do not develop any symptoms and do not feel unwell. The disease can spread through respiratory droplets produced when an infected person coughs or sneezes. These droplets land on objects and surfaces around the person. Other people may acquire SARS-CoV-2 by touching these objects or surfaces, then touching their eyes, nose, or mouth.

Person to person spread was subsequently reported worldwide. The World Health Organization (WHO) has designated the pandemic of COVID-19 a Public Health Emergency of International Concern.

Currently there are no approved treatments for COVID-19 in Europe or the United States. The lack of disease-directed therapeutic options has led to urgent interventions in anticipation of some potentially promising effects. Some antivirals are currently under evaluation. These include favipirivir (AVIGAN) manufactured by Fujifilm in Japan, remdesivir manufactured by Gilead, and Kaletra® (lopinavir/ritonavir) commercially available for human immunodeficiency virus (HIV). There are also investigations of chloroquine and hydroxychloroquine as treatment modalities and potential applications for post-exposure prophylaxis according to Clinicaltrials.gov and other clinical trial registries. These and other potential therapeutic agents are described on the World Health Organization (WHO) website file: WHO Landscape Therapeutics under investigation 17 Feb. 2020.pdf (accessed 19 Mar. 2020).

Accordingly, there is a need for methods and compositions for effectively treating COVID-19 in patients in need thereof, that can reduce all-cause mortality in requiring or not intensive care unit (ICU) admission and that can reduce clinical severity, duration of hospital and ICU stay, dependency of oxygen and ventilator support.

The inventors of the present application have surprisingly discovered that the use of IVIG may be therapeutically beneficial for the treatment of COVID-19 in patients in need thereof. The inventors of the present application have also surprisingly discovered that the use of plasma from convalescent anti-SARS-CoV-2 patients pretreated with methylene blue (MBT) may be therapeutically beneficial. The therapeutic use of convalescent plasma for COVID-19 is also interesting for patients with severe clinical disease for reducing their symptoms, morbidity, and mortality.

To date, a number of possible mechanisms for the immunomodulatory and anti-inflammatory effects of IVIG therapy have been described (Kazatchkine and Kaveri, 2001; Wu et al., 2006), including anti-complement effects (Farbu et al., 2007), anti-idiotypic neutralization of pathogenic autoantibodies (Fernandez-Cruz et al., 2009), immune regulation via an inhibitory Fc receptor (Jordan et al., 2009; Ballow et al., 2011), enhancement of regulatory T cells (Andrew et al., 2011) and inhibition of T helper 17 cells (Th17) differentiation (Akio Matsuda et al., 2012). Thus, IVIG can mediate a wide variety of biological and immunomodulatory effects via various types of blood cells (Akio Matsuda et al., 2012). As such, high dose IVIG may provide therapeutic benefit in the current COVID-19 pandemic.

Fu and colleagues 2020 indicated that potential therapeutic tools to reduce SARS-CoV-2-induced inflammatory responses include various methods to block Fc receptors (FcR) activation. In the absence of a proven clinical FcR blocker, the use of IVIG to block FcR activation may be a viable option for the urgent treatment of pulmonary inflammation to prevent severe lung injury (Fu et al., 2020).

Therefore, high dose of conventional IVIG may be potentially beneficial for the COVID-19 epidemic patients. This may possibly be due to immunomodulatory effects, best evidenced clinically at higher doses.

### SUMMARY

The present disclosure provides methods and compositions for the treatment of COVID-19 in a patient in need thereof. In some embodiments, the method comprises administering to the patient a therapeutically effective amount of intravenous Immunoglobulin G (IVIG) in an amount of about 0.5 g/kg to about 8 g/kg. Therefore, therapeutically doses of IVIG will be administered to those patients hospitalized with COVID-19 in an effort to reduce their symptoms and improve outcomes by leveraging the immunomodulatory effects of IVIG.

In some embodiments, the patient is also subjected to a standard medical treatment (SMT) for COVID-19.

In some embodiments, the IVIG is administered in an amount of about 1 g/kg to about 3 g/kg. Preferably, the IVIG is administered in an amount of about 2 g/kg.

In some embodiments, the IVIG is administered in divided doses. In some embodiments, the IVIG is administered at a dose between 200 mg/dose to 700 mg/dose. Preferably, the IVIG is administered at a dose between 300 mg/dose and 600 mg/dose.

In some embodiments, the IVIG is administered in divided doses over consecutive days. Preferably, the IVIG is administered over 4 to 5 consecutive days.

In some embodiments, the IVIG is administered at a dose of 500 mg/kg body weight over 4 days.

In some embodiments, the IVIG is administered at a dose of 400 mg/kg body weight over 5 days.

In some embodiments, the COVID-19 is caused by the SARS-CoV-2.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient is intensive care unit (ICU) patient.

In some embodiments, the patient is dependent on high flow oxygen devices or invasive mechanical ventilation.

In some embodiments, the patient is non-critical but hospitalized patient.

On the other hand, the present invention comprises a composition comprising a therapeutically effective amount of intravenous Immunoglobulin G (IVIG) in an amount of about 0.5 g/kg to about 8 g/kg for the treatment of COVID-19 in a patient in need thereof.

In some embodiments, the patient is also subjected to a standard medical treatment (SMT) for COVID-19.

In some embodiments, the IVIG is administered in an amount of about 1 g/kg to about 3 g/kg. Preferably, the IVIG is administered in an amount of about 2 g/kg.

In some embodiments, the IVIG is administered in divided doses. In some embodiments, the IVIG is administered at a dose between 200 mg/dose to 700 mg/dose. Preferably, the IVIG is administered at a dose between 300 mg/dose and 600 mg/dose.

In some embodiments, the IVIG is administered in divided doses over consecutive days. Preferably, the IVIG is administered over 4 to 5 consecutive days.

In some embodiments, the IVIG is administered at a dose of 500 mg/kg body weight over 4 days.

In some embodiments, the IVIG is administered at a dose of 400 mg/kg body weight over 5 days.

In some embodiments, the COVID-19 is caused by the SARS-COV-2 virus.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art.

In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient is intensive care unit (ICU) patient.

In some embodiments, the patient is dependent on high flow oxygen devices or invasive mechanical ventilation.

In some embodiments, the patient is non-critical but hospitalized patient.

In a third aspect, the present invention relates to methods for the treatment of coronavirus disease 2019 (COVID-19) in a patient in need thereof, comprising administering to the patient a therapeutically effective amount of convalescent anti-SARS-CoV-2 plasma, wherein the convalescent anti-SARS-CoV-2 plasma is treated with methylene blue for pathogen inactivation.

In some embodiments, the patient is also subjected to standard medical treatment (SMT) for COVID-19.

In some embodiments the methods of the present invention are directed to patients having COVID-19, wherein COVID-19 is mild, moderate or severe.

In some embodiments of the methods of the present invention the patient is positive for SARS-CoV-2 infection as determined by any nucleic acid technology (NAT) or any other commercial or public health assay.

In some embodiments of the methods of the present invention the therapeutically effective amount of convalescent anti-SARS-CoV-2 plasma is between 200 ml to 700 ml of convalescent plasma. In other embodiments the therapeutically effective amount of convalescent anti-SARS-CoV-2 plasma is between 300 ml to 600 ml of convalescent plasma. In other embodiments the therapeutically effective amount of convalescent anti-SARS-CoV-2 plasma is between 400 ml to 500 ml of convalescent plasma. In yet other embodiments, the therapeutically effective amount of convalescent anti-SARS-CoV-2 plasma is between 5 ml to 20 ml of convalescent plasma per kilogram of body weight.

In some embodiments of the methods of the present invention the convalescent anti-SARS-CoV-2 plasma is obtained from more than one convalescent donor.

In some embodiments of the methods of the present invention the therapeutically effective amount of convalescent anti-SARS-CoV-2 plasma is administrated to the patient via intravenous (IV) infusion.

In other embodiments of the methods of the present invention the therapeutically effective amount of convalescent anti-SARS-CoV-2 plasma is administrated to the patient in two or more consecutive intravenous (IV) infusions. In some preferred embodiments, each intravenous infusion consists of 100 ml to 350 ml of convalescent anti-SARS-CoV-2 plasma. In other preferred embodiments, each intravenous infusion consists of 150 ml to 300 ml of convalescent anti-SARS-CoV-2 plasma. In more preferred embodiments, each intravenous infusion consists of 200 ml to 250 ml of convalescent anti-SARS-CoV-2 plasma. In some embodiments the two or more consecutive intravenous (IV) infusions of convalescent anti-SARS-CoV-2 plasma are administrated to the patient the same day. In other embodiments the two or more consecutive intravenous (IV) infusions of convalescent anti-SARS-CoV-2 plasma are administrated to the patient at least every 2 hours, or at least every 4 hours, or at least every 6 hours, or at least every 12 hours, or at least every 24 h, or at least every 48 hours, or at least every 72 hours, or at least once a week, or at least once every two weeks.

In other embodiments of the methods of the present invention the patient requires ICU admission.

In a fourth aspect, the present invention relates to compositions comprising a therapeutically effective amount of convalescent anti-SARS-CoV-2 plasma for use in the treatment of COVID-19 in a patient in need thereof, wherein the convalescent anti-SARS-CoV-2 plasma is treated with methylene blue for pathogen inactivation.

In some embodiments, the patient is also subjected to standard medical treatment (SMT) for COVID-19.

In some embodiments the compositions for use of the present invention are directed to patients having COVID-19, wherein COVID-19 is mild, moderate or severe.

In some embodiments of the compositions for use of the present invention, the patient is positive for SARS-CoV-2 infection as determined by any nucleic acid technology (NAT) or any other commercial or public health assay.

In some embodiments of the compositions for use of the present invention the therapeutically effective amount of convalescent anti-SARS-CoV-2 plasma is between 200 ml to 700 ml of convalescent plasma. In other embodiments the therapeutically effective amount of convalescent anti-SARS-CoV-2 plasma is between 300 ml to 600 ml of convalescent plasma. In other embodiments the therapeutically effective amount of convalescent anti-SARS-CoV-2 plasma is between 400 ml to 500 ml of convalescent plasma. In yet other embodiments, the therapeutically effective amount of convalescent anti-SARS-CoV-2 plasma is between 5 ml to 20 ml of convalescent plasma per kilogram of body weight.

In some embodiments of the compositions for use of the present invention the convalescent anti-SARS-CoV-2 plasma is obtained from more than one convalescent donor.

In some embodiments of the compositions for use of the present invention the therapeutically effective amount of convalescent anti-SARS-CoV-2 plasma is administrated to the patient via intravenous (IV) infusion.

In other embodiments of the compositions for use of the present invention the therapeutically effective amount of convalescent anti-SARS-CoV-2 plasma is administrated to the patient in two or more consecutive intravenous (IV) infusions. In some preferred embodiments each intravenous infusion consists of 100 ml to 350 ml of convalescent anti-SARS-CoV-2 plasma. In other preferred embodiments each intravenous infusion consists of 150 ml to 300 ml of convalescent anti-SARS-CoV-2 plasma. In more preferred embodiments each intravenous infusion consists of 200 ml to 250 ml of convalescent anti-SARS-CoV-2 plasma. In some embodiments, the two or more consecutive intravenous (IV) infusions of convalescent anti-SARS-CoV-2 plasma are administrated to the patient the same day. In other embodiments, the two or more consecutive intravenous (IV) infusions of convalescent anti-SARS-CoV-2 plasma are administrated to the patient at least every 2 hours, or at least every 4 hours, or at least every 6 hours, or at least every 12 hours, or at least every 24 h, or at least every 48 hours, or at least every 72 hours, or at least once a week, or at least once every two weeks.

In other embodiments of the composition for use of the present invention the patient requires ICU admission.

### DETAILED DESCRIPTION

### Definitions

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

In the context of the present invention, the term “disease progression” is defined as the worsening of a subject's condition attributable to the disease for which the patient is been treated. It may be an increase in the severity of the targeted disease and/or increases in the symptoms of the targeted disease. Anticipated symptoms of COVID-19 include fever, cough, hypoxia, dyspnea, hemoptysis, myalgia, fatigue, pharyngitis, which may develop at any time during the course of the disease.

The term “treatment” or “treating” means any treatment of a disease or disorder in a subject, such as a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein.

The term “therapeutically effective amount” refers to that amount of IVIG, typically delivered as pharmaceutical compositions, that is sufficient to effect treatment, as defined herein, when administered to a subject in need of such treatment or an amount of convalescent anti-SARS-CoV-2 plasma that is sufficient to effect treatment, as defined herein, when administered to a subject in need of such treatment.

The term “Nucleic acid technology or NAT”, as used herein, refers to any amplification-based or transcription-based method for detection and quantitation of a target nucleic acid. Numerous amplification-based methods are well known and established in the art, such as PCR, its variation RT-PCR, strand displacement amplification (SDA), thermophilic SDA (tSDA), rolling circle amplification (RCA), helicase dependent amplification (HDA), or loop-mediated isothermal amplification (LAMP). Transcription-based amplification methods commonly used in the art include nucleic acid sequence based amplification (NASBA), Qβ replicase, self-sustained sequence replication or transcription-mediated amplification (TMA).

The term “Methylene Blue Treated (MBT)” as used herein refers to the treatment or pretreatment of a sample with methylene blue for pathogen inactivation. The skilled person is aware of the methods and conditions for the treatment of a sample with methylene blue for pathogen inactivation. In some preferred embodiments, the sample treated with methylene blue is a human sample. In some preferred embodiments the human sample is human blood sample. In more preferred embodiments, the blood sample is plasma, more preferably fresh frozen plasma. It is also contemplated in the context of the present invention that plasma from different donors is pooled prior or after methylene blue treatment.

The term “convalescent plasma” as used herein in refers to plasma collected from previously infected individuals. Thus, the term “convalescent anti-SARS-CoV-2 plasma” as used herein refers to convalescent plasma collected from individuals previously infected with SARS-CoV-2 that have recovered from COVID-19.

In some embodiments, the term “convalescent anti-SARS-CoV-2 MBT plasma” is used for referring to convalescent anti-SARS-CoV-2 plasma previously treated with methylene blue for pathogen inactivation.

The term “standard medical treatment (SMT)” as used herein refers to a treatment that is accepted by medical experts as a proper treatment for a certain type of disease, and that is widely used by healthcare professionals. In the context of the present invention, SMT refers to the standard treatment for COVID-19 patients that is been used in the medical centre where the treatment with convalescent anti-SARS-CoV-2 MBT plasma of the present invention is used. Thus, STM may include treatments with some of the potential therapeutic agents described on the World Health Organization but it may also include other treatments not accepted by the WHO.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

Intravenous Immunoglobulin G (IVIG)

Intravenous immunoglobulin G (IVIG) is the most useful therapeutic indications, whether for the treatment of primary immunodeficiencies or for variable common immunodeficiency (deficit of IgG and IgA subclasses) (Espanol, T. “Primary immunodeficiencies”. Pharmaceutical Policy and Law 2009; 11(4): 277-283), secondary or acquired immunodeficiencies (for example infection by viruses such as cytomegalovirus, herpes zoster, human immunodeficiency) and diseases of an autoimmune origin (thrombocytopenic purpura, Kawasaki's Syndrome, for example) (Koski, C. “Immunoglobulin use in management of inflammatory neuropathy”. Pharmaceutical Policy and Law 2009; 11(4): 307-315).

One suitable example of a pharmaceutical product of IVIG is commercialized under the trade name Flebogamma DIF (Grifols S.A., Spain).

Standard Medical Treatment (SMT)

The term “standard medical treatment (SMT)” as used herein refers to a treatment that is accepted by medical experts as a proper treatment for a certain type of disease, and that is widely used by healthcare professionals. In the context of the present invention, SMT refers to the standard treatment for COVID-19 patients that is been used in the medical centre where the treatment with IVIG of the present invention is used. Thus, STM may include treatments with some of the potential therapeutic agents described on the World Health Organization but it may also include other treatments not accepted by the WHO.

In a first aspect, the present invention relates to methods and compositions for the treatment of COVID-19 in a patient in need thereof. In some embodiments, the method comprises administering to the patient a therapeutically effective amount of intravenous Immunoglobulin G (IVIG) in an amount of about 0.5 g/kg to about 8 g/kg. Therefore, therapeutically doses of IVIG will be administered to those patients hospitalized with COVID-19 in an effort to reduce their symptoms and improve outcomes by leveraging the immunomodulatory effects of IVIG.

In some embodiments, the patient is also subjected to a standard medical treatment (SMT) for COVID-19.

In some embodiments, the IVIG is administered in an amount of about 1 g/kg to about 3 g/kg. Preferably, the IVIG is administered in an amount of about 2 g/kg.

In some embodiments, the IVIG is administered in divided doses. In some embodiments, the IVIG is administered at a dose between 200 mg/dose to 700 mg/dose. Preferably, the IVIG is administered at a dose between 300 mg/dose and 600 mg/dose.

In some embodiments, the IVIG is administered in divided doses over consecutive days. Preferably, the IVIG is administered over 4 to 5 consecutive days.

In some embodiments, the IVIG is administered at a dose of 500 mg/kg body weight over 4 days.

In some embodiments, the IVIG is administered at a dose of 400 mg/kg body weight over 5 days.

In some embodiments, the COVID-19 is caused by the SARS-CoV-2.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient is intensive care unit (ICU) patient.

In some embodiments, the patient is dependent on high flow oxygen devices or invasive mechanical ventilation.

In some embodiments, the patient is non-critical but hospitalized patient.

In a second aspect, the present invention relates to a composition comprising a therapeutically effective amount of intravenous Immunoglobulin G (IVIG) in an amount of about 0.5 g/kg to about 8 g/kg for the treatment of COVID-19 in a patient in need thereof.

In some embodiments, the patient is also subjected to a standard medical treatment (SMT) for COVID-19.

In some embodiments, the IVIG is administered in an amount of about 1 g/kg to about 3 g/kg. Preferably, the IVIG is administered in an amount of about 2 g/kg.

In some embodiments, the IVIG is administered in divided doses. In some embodiments, the IVIG is administered at a dose between 200 mg/dose to 700 mg/dose. Preferably, the IVIG is administered at a dose between 300 mg/dose and 600 mg/dose.

In some embodiments, the IVIG is administered in divided doses over consecutive days. Preferably, the IVIG is administered over 4 to 5 consecutive days.

In some embodiments, the IVIG is administered at a dose of 500 mg/kg body weight over 4 days.

In some embodiments, the IVIG is administered at a dose of 400 mg/kg body weight over 5 days.

In some embodiments, the COVID-19 is caused by the SARS-COV-2 virus.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient is intensive care unit (ICU) patient.

In some embodiments, the patient is dependent on high flow oxygen devices or invasive mechanical ventilation.

In some embodiments, the patient is non-critical but hospitalized patient.

In a third aspect, the present invention relates to methods for the treatment of coronavirus disease 2019 (COVID-19) in a patient in need thereof, comprising administering to the patient a therapeutically effective amount of convalescent anti-SARS-CoV-2 plasma.

Convalescent anti-SARS-CoV-2 plasma may be obtained from a donor recovered from COVID-19 of from various donors recovered from COVID-19.

In some embodiments, the convalescent anti-SARS-CoV-2 plasma is treated for pathogen inactivation. In more preferred embodiments, the convalescent anti-SARS-CoV-2 plasma is treated with methylene blue for pathogen inactivation. The skilled person is aware of the methods and conditions for the treatment of a sample, such as plasma, with methylene blue for pathogen inactivation. In some embodiments, the convalescent plasma is treated with methylene blue right after it is obtained from the pa donor. In other embodiments, the convalescent plasma is treated with methylene blue in a later stage. In other embodiments, a pool of convalescent plasma from different donors is treated with methylene blue at any stage.

Thus, in some embodiments, present invention relates to methods for the treatment of coronavirus disease 2019 (COVID-19) in a patient in need thereof, comprising administering to the patient a therapeutically effective amount of convalescent anti-SARS-CoV-2 plasma, wherein the convalescent anti-SARS-CoV-2 plasma is treated with methylene blue for pathogen inactivation.

In some embodiments of the methods of the present invention, the patient is also subjected to standard medical treatment (SMT) for COVID-19. STM refers to the standard treatment for COVID-19 patients that is been used in the medical centre where the treatment with convalescent anti-SARS-CoV-2 MBT plasma is used.

The methods of the present invention are directed to patients having COVID-19, wherein COVID-19 is mild, moderate or severe.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient of the present invention is a hospitalized male or female patients of >=18 years of age with positive result for SARS-CoV-2 by any of the above technique less than 72 hours prior to being administrated with a therapeutically effective amount of convalescent anti-SARS-CoV-2 plasma. In some embodiments, the patient is positive for SARS-CoV-2 by any of the above technique less than 5 days prior to being administrated with a therapeutically effective amount of convalescent anti-SARS-CoV-2 plasma.

The method of the present invention comprises administering to the patient a therapeutically effective amount of convalescent anti-SARS-CoV-2 plasma.

In some embodiments the therapeutically effective amount of convalescent anti-SARS-CoV-2 plasma is between 200 ml to 700 ml of convalescent plasma. In more preferred embodiments, the therapeutically effective amount of convalescent anti-SARS-CoV-2 plasma is between 300 ml to 600 ml of convalescent plasma. In more preferred embodiments, the therapeutically effective amount of convalescent anti-SARS-CoV-2 plasma is between 400 ml to 500 ml of convalescent plasma.

In other embodiments, the therapeutically effective amount of convalescent anti-SARS-CoV-2 plasma is between 5 ml to 20 ml of convalescent plasma per kilogram of body weight. In more preferred embodiments, the therapeutically effective amount of convalescent anti-SARS-CoV-2 plasma is between 10 ml to 15 ml of convalescent plasma per kilogram of body weight. In even more preferred embodiments, the therapeutically effective amount of convalescent anti-SARS-CoV-2 plasma is about 10 ml of convalescent plasma per kilogram of body weight.

In some embodiments of the method of the present invention the convalescent anti-SARS-CoV-2 plasma is obtained from more than one convalescent donor. In some preferred embodiments, the convalescent anti-SARS-CoV-2 plasma is obtained from at least two convalescent donors, or at least three convalescent donors, or at least five convalescent donors.

In some embodiments, the therapeutically effective amount of convalescent anti-SARS-CoV-2 plasma is administrated to the patient via intravenous (IV) infusion. In some embodiments, the therapeutically effective amount of convalescent anti-SARS-CoV-2 plasma is administrated to the patient in two or more consecutive intravenous (IV) infusions. In the embodiments in which the therapeutically effective amount of convalescent anti-SARS-CoV-2 plasma is administrated to the patient in two or more consecutive intravenous (IV) infusions the convalescent anti-SARS-CoV-2 plasma can be obtained from one donor or from more than one donor.

In some preferred embodiments in which the convalescent anti-SARS-CoV-2 plasma is administrated to the patient in two or more consecutive intravenous (IV) infusions, each intravenous infusion consists of 100 ml to 350 ml of convalescent anti-SARS-CoV-2 plasma. More preferably each intravenous infusion consists of 150 ml to 300 ml of convalescent anti-SARS-CoV-2 plasma. Even more preferably, each intravenous infusion consists of 200 ml to 250 ml of convalescent anti-SARS-CoV-2 plasma.

In some embodiments, the two or more consecutive intravenous (IV) infusions of convalescent anti-SARS-CoV-2 plasma are administrated to the patient the same day. In other embodiments, the two or more consecutive intravenous (IV) infusions of convalescent anti-SARS-CoV-2 plasma are administrated to the patient at least every 2 hours, or at least every 4 hours, or at least every 6 hours, or at least every 12 hours, or at least every 24 h, or at least every 48 hours, or at least every 72 hours, or at least once a week, or at least once every two weeks.

In some embodiments, the present invention relates to methods for the treatment of coronavirus disease 2019 (COVID-19) in a patient in need thereof, wherein the patient requires ICU admission. In some embodiments, the patient has being treated in the intensive care unit (ICU) for COVID-19 for not longer than 48 hours. In other embodiments, the patient is a patient for whom a decision is made that COVID-19 disease severity warrants ICU admission.

In a fourth aspect, the present invention relates to compositions comprising a therapeutically effective amount of convalescent anti-SARS-CoV-2 plasma for use in the treatment of COVID-19 in a patient in need thereof.

Convalescent anti-SARS-CoV-2 plasma may be obtained from a donor recovered from COVID-19 of from various donors recovered from COVID-19.

In some embodiments, the convalescent anti-SARS-CoV-2 plasma is treated for pathogen inactivation. In more preferred embodiments, the convalescent anti-SARS-CoV-2 plasma is treated with methylene blue for pathogen inactivation. The skilled person is aware of the methods and conditions for the treatment of a sample, such as plasma, with methylene blue for pathogen inactivation. In some embodiments, the convalescent plasma is treated with methylene blue right after it is obtained from the pa donor. In other embodiments, the convalescent plasma is treated with methylene blue in a later stage. In other embodiments, a pool of convalescent plasma from different donors is treated with methylene blue at any stage.

Thus, in some embodiments, present invention relates to compositions comprising a therapeutically effective amount of convalescent anti-SARS-CoV-2 plasma for use in the treatment of COVID-19 in a patient in need thereof, wherein the convalescent anti-SARS-CoV-2 plasma is treated with methylene blue for pathogen inactivation.

In some embodiments of the composition for use of the present invention, the patient is also subjected to standard medical treatment (SMT) for COVID-19. STM refers to the standard treatment for COVID-19 patients that is been used in the medical centre where the treatment with convalescent anti-SARS-CoV-2 MBT plasma is used.

The compositions for use of the present invention are directed to patients having COVID-19, wherein COVID-19 is mild, moderate or severe.

In some embodiments of the compositions for use of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient of the present invention is a hospitalized male or female patients of >=18 years of age with positive result for SARS-CoV-2 by any of the above technique less than 72 hours prior to being administrated with a therapeutically effective amount of convalescent anti-SARS-CoV-2 plasma. In some embodiments, the patient is positive for SARS-CoV-2 by any of the above technique less than 5 days prior to being administrated with a therapeutically effective amount of convalescent anti-SARS-CoV-2 plasma.

In some embodiments the therapeutically effective amount of convalescent anti-SARS-CoV-2 plasma is between 200 ml to 700 ml of convalescent plasma. In more preferred embodiments, the therapeutically effective amount of convalescent anti-SARS-CoV-2 plasma is between 300 ml to 600 ml of convalescent plasma. In more preferred embodiments, the therapeutically effective amount of convalescent anti-SARS-CoV-2 plasma is between 400 ml to 500 ml of convalescent plasma.

In other embodiments, the therapeutically effective amount of convalescent anti-SARS-CoV-2 plasma is between 5 ml to 20 ml of convalescent plasma per kilogram of body weight. In more preferred embodiments, the therapeutically effective amount of convalescent anti-SARS-CoV-2 plasma is between 10 ml to 15 ml of convalescent plasma per kilogram of body weight. In even more preferred embodiments, the therapeutically effective amount of convalescent anti-SARS-CoV-2 plasma is about 10 ml of convalescent plasma per kilogram of body weight.

In some embodiments of the composition for use of the present invention the convalescent anti-SARS-CoV-2 plasma is obtained from more than one convalescent donor. In some preferred embodiments, the convalescent anti-SARS-CoV-2 plasma is obtained from at least two convalescent donors, or at least three convalescent donors, or at least five convalescent donors.

In some embodiments, the therapeutically effective amount of convalescent anti-SARS-CoV-2 plasma is administrated to the patient via intravenous (IV) infusion. In some embodiments, the therapeutically effective amount of convalescent anti-SARS-CoV-2 plasma is administrated to the patient in two or more consecutive intravenous (IV) infusions. In the embodiments in which the therapeutically effective amount of convalescent anti-SARS-CoV-2 plasma is administrated to the patient in two or more consecutive intravenous (IV) infusions the convalescent anti-SARS-CoV-2 plasma can be obtained from one donor or from more than one donor.

In some preferred embodiments in which the convalescent anti-SARS-CoV-2 plasma is administrated to the patient in two or more consecutive intravenous (IV) infusions, each intravenous infusion consists of 100 ml to 350 ml of convalescent anti-SARS-CoV-2 plasma. More preferably each intravenous infusion consists of 150 ml to 300 ml of convalescent anti-SARS-CoV-2 plasma. Even more preferably, each intravenous infusion consists of 200 ml to 250 ml of convalescent anti-SARS-CoV-2 plasma.

In some embodiments, the two or more consecutive intravenous (IV) infusions of convalescent anti-SARS-CoV-2 plasma are administrated to the patient the same day. In other embodiments, the two or more consecutive intravenous (IV) infusions of convalescent anti-SARS-CoV-2 plasma are administrated to the patient at least every 2 hours, or at least every 4 hours, or at least every 6 hours, or at least every 12 hours, or at least every 24 h, or at least every 48 hours, or at least every 72 hours, or at least once a week, or at least once every two weeks.

In some embodiments, the present invention relates to compositions comprising a therapeutically effective amount of convalescent anti-SARS-CoV-2 plasma for use in the treatment of COVID-19 in a patient in need thereof. In some embodiments, said patient requires ICU admission. In other embodiments, the patient has being treated in the intensive care unit (ICU) for COVID-19 for not longer than 48 hours. In other embodiments, the patient is a patient for whom a decision is made that COVID-19 disease severity warrants ICU admission.

### EXAMPLES

### Example 1. Selection of Plasma Donors for Collection of SARS-CoV-2 Convalescent Plasma

For the selection of plasma donors for obtaining SARS-CoV-2 convalescent plasma for use in the production of the hyperimmune globule composition of the present invention, the method described in U.S. 63/034,289 (incorporated by reference herein) is used.

In brief, individuals in good health who have been approved through the pre-screening process are allowed to proceed to the donation center for final evaluation and donation. This pre-screening process assured that only individuals who have recovered from their illness, or were exposed to the disease agent but remained asymptomatic, would qualify to come into the center and potentially donate. Thus, only individuals that had a laboratory evidence of COVID-19 infection, either through nucleic acid amplification testing (NAT), positive antigen test, or by SARS-CoV-2 antibody test prior to enrollment, and were then in a convalescent noninfectious state may be safely processed within the donor center.

Thus, symptomatic donors had to have complete resolution of symptoms at least 14 days before the donation if they were negative by a follow-up NAT, or 28 days if they had no follow-up test. Similarly, asymptomatic donors who were positive by NAT or antigen tests were required to wait 14 days after the initial test if they had a follow-up negative NAT, but had to wait 28 days after the initial test if they had no follow-up test. Asymptomatic donors who were only tested by an anti-SARS-CoV-2 antibody test were required to wait seven days prior to donation, but could donate immediately if they also had a negative NAT.

Donors also had to be negative for human leukocyte antigen (HLA) antibodies.

Table 1 summarizes the above criteria for plasma donors' eligibility based on symptoms and test results.

TABLE 1
Criteria for plasma donors' eligibility.

| | Test results | 1^{st} Eligible date |
|---|---|---|
| Symptomatic | No NAT, Negative | Not eligible, but could be |
| | Antibody | eligible for normal source |
| | | plasma |
| | Positive NAT only | 28 days after cessation of all |
| | Positive NAT with | symptoms. |
| | subsequent Positive | NOTE: The Positive Antibody |
| | Antibody | test indicates past infection |
| | Positive Antibody | with COVID 19; |
| | only | |
| | Positive NAT, | |
| | Antibody negative | |
| | Positive NAT, then | 28 days after cessation of all |
| | second Positive NAT | symptoms or 28 days after |
| | later | second positive at NAT test, |
| | | whichever is later |
| | Positive NAT with | 14 days after cessation of all |
| | subsequent Negative | symptoms. |
| | NAT | NOTE: The second negative |
| | | NAT test indicates that |
| | | the donor no longer has a |
| | | detectable amount of virus and |
| | | therefore the deferral period |
| | | may be shortened. |
| Asymptomatic | No NAT, negative | Eligible for Normal Source |
| | antibody | Plasma |
| | Negative NAT and | Eligible today |
| | Positive Antibody | |
| | if collected at | |
| | same time | |
| | Positive NAT only | 28 days after date of positive |
| | Positive NAT with | at NAT sample. Confirmation |
| | subsequent Positive | needed that subsequently, no |
| | Antibody | symptoms were developed. If |
| | | symptoms developed, reset the |
| | | calendar to a symptom-based. |
| | | NOTE: The Positive Antibody |
| | | test indicates past infection |
| | | with COVID 19 |
| | Positive Antibody | At least 7 days from the |
| | only | serology test result if no |
| | | symptoms were developed. |
| | Positive NAT with | 14 days after date of positive |
| | subsequent Negative | at NAT sample. |
| | NAT | NOTE: The second negative |
| | | NAT test indicates that the |
| | | donor no longer has a |
| | | detectable amount of virus and |
| | | therefore the deferral period |
| | | may be shortened. |
| | Positive antigen | 28 days after date of positive at |
| | only | NAT sample. Confirmation |
| | | needed that subsequently, no |
| | | symptoms were developed. |
| | Positive antigen | 14 days after date of positive |
| | with subsequent | at NAT sample. |
| | Negative NAT | |

### Example 2. Manufacture of SARS-CoV-2 Convalescent Human Plasma

Once the donor has been selected as explained in example 1 or following any other criteria, plasma is collected by plasmapheresis.

Each plasma unit must meet requirements for source plasma for manufacturing as defined by regulations including screening against a variety of infectious agents. Additionally, each unit was tested to confirm it was negative for SARS-CoV-2 virus and positive for anti-SARS-CoV-2 antibodies.

Each plasma sample was also tested to be negative for human leukocyte antigen (HLA) antibodies and blood typed. Then, plasma pools were modeled to maintain consistent distribution with the overall donor ABO blood type distribution to maintain consistent batch to batch levels of anti-A and anti-B.

These parameters are normally limited by dilution when large batches of plasma are pooled together to make immunoglobulin products, but with smaller batches, single donors could have a greater influence on the final product.

Thus, type O and Type B donors were limited to no more than two units from any single donor for each plasma pool to decrease the likelihood of having high anti-A titers in the final product.

In this example, the ABO blood typing results from 500 plasma units used for manufacturing the pool batches of SARS-CoV-2 convalescent human plasma are presented in Table 2. Results from two published studies are included as comparators. These results show that ABO blood type distribution for the COVID-19 convalescent plasma donors was similar to the distributions reported in other studies of blood and plasma donors.

TABLE 2
ABO Blood type distribution of convalescent plasma from test
batches for this invention compared to published values.

ABO Type (%)

| | A | B | O | AB |
|---|---|---|---|---|
| This invention (500 Units) | 36.4 | 9.0 | 48.6 | 6.0 |
| Garratty et al, (2004) | 37.1 | 12.2 | 46.6 | 4.1 |
| McVey et al (2015) | 38.0 | 11.3 | 47.1 | 3.7 |

### Example 3. Manufacture of a Liquid Therapeutic Hyperimmune Globulin Composition from SARS-CoV-2 Convalescent Plasma

The plasma pools obtained in the example 2 were then processed following the same steps as the Gamunex-C caprylate/chromatography process (Lebing, W., et al., 2003, U.S. Pat. No. 6,307,028, each incorporated by reference herein), which included multiple steps validated for the removal and/or inactivation of viruses (Gamunex-C [Immune Globulin Injection (Human) 10% Caprylate/Chromatography Purified]-Package Insert. 2020).

The resulting product was a highly purified IgG solution (SARS-CoV-2 human immunoglobulin (hIVIG)) formulated at around 10% protein content with glycine at a low pH.

### Example 4. Characterization of SARS-CoV-2 Human Immunoglobulin (hIVIG) Product

The hyperimmune globulin composition of the present invention (hIVIG), obtained from SARS-CoV-2 convalescent human plasma, was characterized to assess the recovery of anti-SARS-CoV-2 specific antibodies. Thus, hIVIG product was tested with an IgG specific Enzyme-linked immunosorbent assay (ELISA) and a neutralizing antibody assay.

Characterization of hIVIG product also included prior routine batch testing to characterize the product and ascertain that it is suitable for use. This characterization included analyses for glycine, pH, protein concentration, osmolality, composition by electrophoresis, and molecular weight profiling by size exclusion chromatography. Analyses were also performed for sodium caprylate, residual IgA and IgM, prekallikrein activator (PKA), factor Xa, anti-A, anti-B, and anti-D. In addition, compendial tests for sterility and pyrogenic substances were performed on all batches.

These tests showed that the tested batches were within the batch standards for purity, formulation, molecular profile and purity described for other immune globulin products manufactured with the caprylate/chromatography process, such as Gamunex-C. The batches also passed USP pyrogen and sterility tests.

Surprisingly, these tests showed that between 97% and 100% of the protein content was IgG. In addition, the IgG was present almost entirely as monomers and dimers with aggregates and fragments below the limits of detection. A process impurity (sodium caprylate) and plasma protein impurities were found at very low concentrations in the final product, well under the batch requirements.

The amounts of residual IgA and IgM were also below the batch requirements (less than 0.13 mg/ml and less than 0.030 mg/ml, respectively) and the concentrations known for the Gamunex-C product.

IgM has been identified as a primary source of anti-A and anti-B intravascular hemolytic activity (Flegel, W. A., 2015). The hIVIG product of the present invention was shown to contain less than 0.01 mg/ml, which greatly reduces the danger of this adverse event. In contrast, when patients are treated with convalescent plasma, they must be matched by donor blood type to reduce the chances of hemolysis.

Similarly, removal of IgA provides a potential therapeutic advantage for hIVIG products over convalescent plasma in patients who are IgA deficient and may have been previously treated with blood products and formed antibodies to IgA. The hIVIG product of the present invention was shown to contain less than 0.04 mg/ml of IgA.

Anti-SARS-CoV-2 ELISA

Anti-SARS-CoV-2 IgG titers were determined using Human Anti-SARS-CoV-2 Virus Spike 1 (51) IgG assay from Alpha Diagnostic. 20 hIVIG batches were tested using multiple serial dilutions and a curve constructed by plotting the log of the optical density as a function of the log of the dilution. The titer was defined as the dilution at which this curve is equal to the low kit standard. The titer was also expressed as a ratio to an in-house control, which consists of a commercially available chimeric monoclonal SARS-CoV-2 S1 antibody (Sino Biologicals, Beijing, China) spiked into plasma from non-COVID-19 donors at levels intended to give titers similar to those found in plasma of COVID-19 donors.

The results are presented in Table 3 for the 20 hIVIG batches produced and its corresponding plasma pool. Said results demonstrated that ELISA activity (ELISA titer, 1:X) increased up to almost 30-fold, when processing the pooled plasma into the final product. The IgG concentration was also increased more than 10-fold from the pooled plasma to the final product. When anti-SARS-CoV-2 antibody titers were normalized to the IgG concentration, data varies between 250 and 2,500, which result in similar values for the starting material and the finished product. This can be explained by contributions from IgM and IgA to the ELISA activity, which have been removed during purification of IgG and demonstrates once again the high purity of the hIVIG final product.

TABLE 3
Anti-SARS-CoV-2 titers and specific activities (n = 20, ±standard deviation).

| | | | | | Normalized |
|---|---|---|---|---|---|
| | | | Normalized | Neutralizing | Neutralizing |
| | | | ELISA | Activity | Activity, |
| Sample | IgG, | ELISA | Titer, titer/ | Titer, | IC50/mg/mL |
| Point | mg/mL | Titer, 1:X | mg/mL IgG | IC50, 1:X | IgG |
| Pool | 8.0 ± 0.2 | 9,038 ± 4037 | 1,131 ± 497 | 110 ± 15 | 13.9 ± 2.2 |
| Final | 100 ± 3 | 25,000-250,000 | 250-2,500 | 150-1,500 | 1.5-15 |
| Product | | | | | |
| (n = 20) | | | | | |

Anti-SARS-CoV-2 Neutralizing Antibody Assay

The hIVIG products were also tested for anti-SARS-CoV-2 antibodies using an immunofluorescence-based neutralization assay performed at the National Institutes of Health Integrated Research Facility, Frederick, Md. This assay quantifies the anti-SARS-CoV-2 neutralization titer by using a dilution series of test material to test for inhibition of infection of cultured Vero (CCL-81) cells by SARS-CoV-2 (Washington isolate, CDC).

Potency was assessed using a cell-based immunosorbent assay to quantify infection by detecting the SARS-CoV-2 nucleoprotein using a specific antibody raised against the SARS-CoV-1 nucleoprotein.

The secondary detection antibody was conjugated to a fluorophore which allows quantification of individual infected cells on a high throughput optical imaging system. A minimum of 16,000 cells were counted per sample dilution across four wells—two each in duplicate plates. Data are reported based on a 4-parameter regression curve (using a constrained fit) as a 50% neutralization titer (1050) in Table 3.

The results showed that antibody neutralizing activity (1050) was increased more than 10-fold from the plasma pool to the final product. This increase in neutralizing activity indicates that patients treated with hIVIG products compared to an equivalent volume of convalescent plasma would receive higher neutralizing activity. Alternatively, patients treated with hIVIG could receive a smaller treatment volume compared to treatment with convalescent plasma and potentially decrease the chances for transfusion-associated circulatory overload.

Specific neutralizing activity (normalized to the IgG concentration), was slightly reduced in final product compared to plasma. As previously discussed, this may be caused by contributions from IgM and IgA which have been removed during purification of IgG.

An advantage of using SARS-CoV-2 convalescent human plasma to manufacture the hIVIG product of the present invention (compared to direct administration of plasma from individuals or administration of a monoclonal antibody) is the diversity of antibodies obtained from a pool of convalescent donors which may provide a wider range of anti-viral activity. This diversity is important in overcoming mutations in the virus. Antibody diversity provides a broader range of anti-viral activity by attacking different viral epitopes and enlisting different cellular mechanisms. Neutralization of free virus is mainly the result of steric blocking to prevent infection, whereas additional anti-viral activity may come from activation of effector functions such as complement-mediated or antibody-dependent cellular cytotoxicity.

1. A method for the treatment of coronavirus disease 2019 (COVID-19) in a patient in need thereof, comprising administering to the patient a therapeutically effective amount of intravenous Immunoglobulin G (IVIG), wherein the IVIG is administered in an amount of about 0.5 g/kg to about 8 g/kg

2. The method according to claim 1, wherein the patient is also subjected to a standard medical treatment (SMT) for COVID-19.

3. The method according to claim 1, wherein the IVIG is administered in an amount of about 1 g/kg to about 3 g/kg.

4. The method according to claim 3, wherein the IVIG is administered in an amount of about 2 g/kg.

5. The method according to claim 1, wherein the IVIG is administered in divided doses.

6. The method according to claim 1, wherein the IVIG is administered at a dose between 200 mg/dose to 700 mg/dose.

7. The method according to claim 6, wherein the IVIG is administered at a dose between 300 mg/dose and 600 mg/dose.

8.-16. (canceled)

17. A composition comprising a therapeutically effective amount of intravenous Immunoglobulin G (IVIG) for the treatment of COVID-19 in a patient in need thereof, wherein the IVIG is administered in an amount of about 0.5 g/kg to about 8 g/kg.

18. The composition according to claim 17, wherein the patient in need thereof is also subjected to a standard medical treatment (SMT) for COVID-19.

19. The composition according to claim 17, wherein the IVIG is administered in an amount of about 1 g/kg to about 3 g/kg.

20. The composition according to claim 19, wherein the IVIG is administered in an amount of about 2 g/kg.

21. The composition according to claim 17, wherein the IVIG is administered in divided doses.

22. The composition according to claim 17, wherein the IVIG is administered at a dose between 200 mg/dose to 700 mg/dose.

23.-49.

50. A composition comprising a therapeutically effective amount of convalescent anti-SARS-CoV-2 plasma for use in the treatment of COVID-19 in a patient in need thereof, wherein the convalescent anti-SARS-CoV-2 plasma is treated with methylene blue for pathogen inactivation.

51. The composition according to claim 50, wherein the patient is also subjected to standard medical treatment (SMT) for COVID-19.

52. The composition according to claim 50, wherein COVID-19 is mild, moderate or severe.

53. The composition according to claim 50, wherein the patient is positive for SARS-CoV-2 infection as determined by any nucleic acid technology (NAT) or any other commercial or public health assay.

54. The composition according to claim 50, wherein the therapeutically effective amount of convalescent anti-SARS-CoV-2 plasma is between 200 ml to 700 ml of convalescent plasma.

55. The composition according to claim 50, wherein the therapeutically effective amount of convalescent anti-SARS-CoV-2 plasma is between 300 ml to 600 ml of convalescent plasma.

56. The composition according to claim 50, wherein the therapeutically effective amount of convalescent anti-SARS-CoV-2 plasma is between 400 ml to 500 ml of convalescent plasma.

57.-66. (canceled)
